(19) United States
(12) Patent Application Publication
(10) Pub. No.: US 2012/0264699 A1
(43) Pub. Date: Oct. 18, 2012

(54) METHOD OF PRODUCING AUTOGENOUS OR ALLOGENIC BLOOD SERUM AND RELATED LOGISTICS

(76) Inventors: Reinhold Schmieding, Naples, FL (US); David O. Shepard, Naples, FL (US)

(21) Appl. No.: 13/532,178

(22) Filed: Jun. 25, 2012

Related U.S. Application Data
(63) Continuation of application No. 11/350,078, filed on Feb. 9, 2006, now abandoned.
(60) Provisional application No. 60/651,063, filed on Feb. 9, 2005.

(51) Int. Cl. A61K 38/02

(52) U.S. Cl. 514/21.92

(57) ABSTRACT

A method of producing blood serum containing prophylactically or therapeutically active proteins, including obtaining blood from a patient, incubating the blood at a suitable temperature to induce production of prophylactically or therapeutically active proteins, and removing the prophylactically or therapeutically active proteins from the blood.

### CROSS-REFERENCE TO RELATED APPLICATIONS

This is a continuation of Ser. No. 11/350,078, filed Feb. 9, 2006; now abandoned, which claims priority from U.S. Provisional Application No. 60/651,063, filed Feb. 9, 2005, the disclosures of which are incorporated herein by reference in their entireties.

### BACKGROUND OF THE INVENTION

1. Field of the Invention

The present invention relates to a method for producing autogenous or allogenic blood serum containing prophylactically or therapeutically active proteins.

2. Description of the Related Art

Methods for producing interleukin-1 receptor antagonist (“IL-1Ra”) from a patient's blood are described in U.S. Pat. Nos. 6,759,188; 6,623,472; and 6,713,246, hereby incorporated by reference in their entirety. However, these methods require the use of a special syringe to produce the IL-1Ra and do not provide methods for dividing the protein serum containing IL-1Ra or other therapeutically active proteins into portions for long-term storage and/or transportation.

Accordingly, a need exists for a method of producing and handling autogenous or allogenic blood serum containing IL-1Ra and/or other prophylactically or therapeutically active proteins without the need for a special syringe.

### SUMMARY OF THE INVENTION

The present invention fulfills the above-described need by providing a method of producing blood serum containing prophylactically or therapeutically active proteins, including the steps of obtaining blood from a patient, incubating the blood at a suitable temperature to induce production of prophylactically or therapeutically active proteins, removing the prophylactically or therapeutically active proteins from the blood, and treating the patient by administering the autogenous or allogenic serum containing the prophylactically or therapeutically active proteins to the patient. Patients suffering from osteoarthritis and tendonitis may benefit from such treatment. In exemplary veterinary medicine applications, serum from a donor herd of horses can be used to produce prophylactically or therapeutically active proteins for administration to other horses in need of treatment.

If the patient is not to be treated immediately with the serum, the invention further provides for dividing the protein serum component and storing it in a container (step 180). The container can be frozen to preserve the serum for long or short term storage and/or for transportation of the serum until the determination is made that the patient should be treated.

### BRIEF DESCRIPTION OF THE DRAWINGS

The foregoing and other advantages and features of the invention will be more readily understood from the following detailed description of the invention provided below with reference to the accompanying drawings, in which:

FIG. 1 is a flowchart of an exemplary method in accordance with the invention; and

FIGS. 2A, 2B, and 2C are cross-sectional views of a sterile container in accordance with the invention.

### DETAILED DESCRIPTION OF THE INVENTION

In the following detailed description, reference is made to the accompanying drawings, which form a part hereof and show by way of illustration specific embodiments in which the invention may be practiced. These embodiments are described in sufficient detail to enable those skilled in the art to practice the invention, and it is to be understood that other embodiments may be utilized, and that structural, logical, and electrical changes may be made without departing from the spirit and scope of the present invention. The progression of processing steps described is exemplary of the embodiments of the invention; however, the sequence of steps is not limited to that set forth herein and may be changed as is known in the art, with the exception of steps necessarily occurring in a certain order.

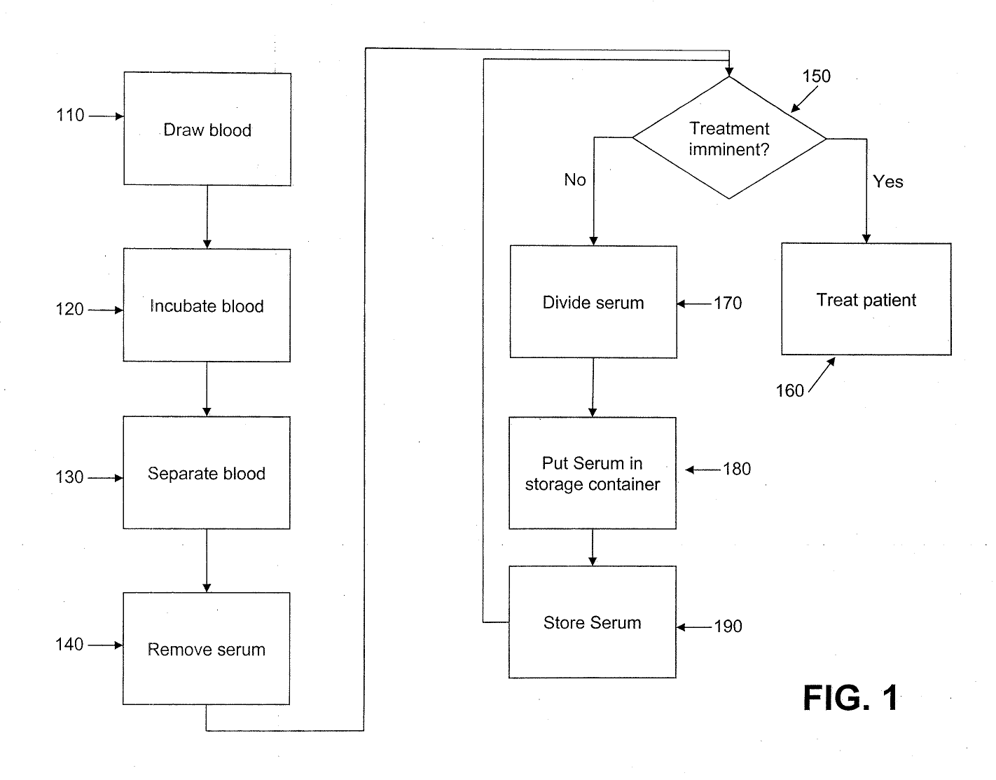

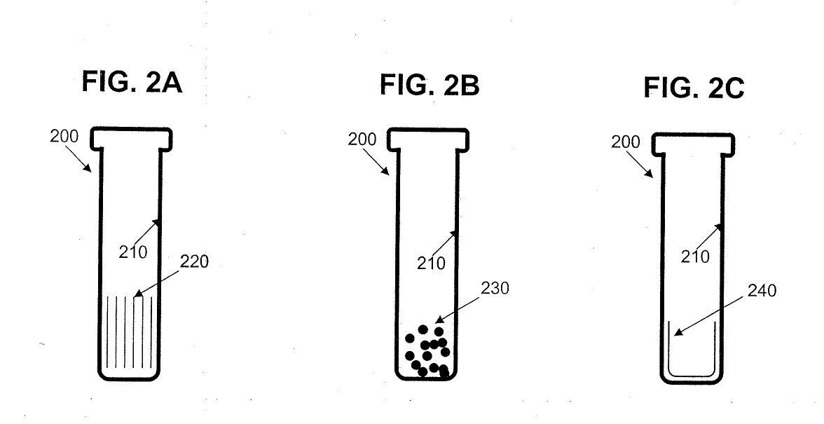

Now referring to the figures, where like numerals designate like elements, FIG. 1 is a flowchart of an exemplary method in accordance with the invention. In this embodiment of the invention, in step 110, blood is obtained from a patient. In step 120, the blood is incubated in a sterile container 200 (FIGS. 2A, 2B, and 2C) at a temperature suitable to induce production of prophylactically or therapeutically active proteins (e.g., IL-1Ra, interleukin 4, interleukin 10, and TGF beta). The sterile container 200 can be of any suitable size or configuration (e.g., vial, capped container, closed tube) and is preferably made of a material that can be subjected to sterilization (e.g., chemical sterilization, autoclave). For example, the container 200 can be made of any suitable glass, ceramic, or plastic material (e.g., polystyrene, polypropylene).

The blood is incubated from about 35 to 39° C., more preferably from about 36° C. to about 38° C., most preferably at 37° C. The blood is incubated for a period of time suitable to produce a sufficient quantity of the therapeutically active protein to treat the patient. The blood is incubated in the sterile container from about 6 to about 36 hours, more preferably from about 10 to about 24 hours, most preferably for about 12 hours.

In step 130, components of the blood are separated following incubation of the blood. In a preferred embodiment, blood cells (e.g., monocytes) are separated from the serum by any suitable method (e.g., centrifugation). In step 140, the protein serum component containing the therapeutically or prophylactically active proteins produced during incubation is removed. A determination is made at step 150 whether treatment of the patient is imminent. If so, at step 160, the patient is treated with the serum. If not, the protein serum component is divided (step 170) and stored in a container (step 180). Then, at step 190, the container is frozen to preserve the serum for long or short term storage and/or for transportation of the serum until the determination is made that the patient should be treated (repeat step 150).

As a specific, but non-limiting example, 10 to 60 cc of blood is drawn (step 110) from the patient and incubated at about 37° C. for about 12 hours in a sterile container having a modified inner surface to increase surface area and monocyte adherence (step 120). The blood is centrifuged to separate the protein serum from the blood cell layer (step 130). The protein serum is then removed (step 140) from the sterile container. Since, in this example, the patient is not to be treated near the time of removal (step 150), the serum is placed, for example, into multiple containers or vials (steps 170 and 180). The serum is deep frozen for storage within about 24 hours (step 190).

Upon a determination that the treatment will be made (step 150), the serum is thawed. After thawing, a portion of the serum (e.g., one container or vial) of serum is administered (e.g., via injection or perfusion) by an orthopedic surgeon into a patient, for example, at a specific site (e.g., specific joint, tendon, muscle, or other soft tissue) to treat or reduce the symptoms associated with a disease condition (e.g., osteoarthritis or tendonitis) (step 160).

FIGS. 2A, 2B, and 2C illustrate a sterile container 200 modified in accordance with the invention. In this example, the sterile container 200 is a test tube. The sterile container 200 is modified in order to increase the surface area of the inner surface 210 of the container 200. For example, as shown in FIG. 2A, the inner surface 210 of the sterile container 200, or a portion thereof, can be treated with a corrosive agent (e.g., acid such as chromosulfonic acid) resulting in the etching 220 of the inner surface 210 of the container 200. Alternatively, as shown in FIG. 2B, the surface area of the inner surface 210 of the sterile container 200, or a portion thereof, can be increased by adding and/or coating the inner surface 210 of the container 200 with granules 230 (e.g., made of glass or plastic) or other suitable materials (e.g., gels, wool, spheres, and particles). Without being bound by theory, it is believed that the increased surface area provides additional attachments for adherence by blood monocytes (not shown), which stimulates the monocytes to produce therapeutically or prophylactically active proteins such as, for example, IL-1Ra. In one embodiment, as shown in FIG. 2C, the inner surface 210 of the container 200, or a portion thereof, can be coated with an anti-coagulant 240 such as heparin.

While the invention has been described in detail in connection with exemplary embodiments known at the time, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention.

Thus, the invention is not to be seen as limited by the foregoing description, but is only limited by the scope of the appended claims.

What is claimed as new and desired to be protected by Letters Patent of the United States is:

1. A method of producing blood serum containing prophylactically or therapeutically active proteins comprising:
obtaining blood from a donor;
transferring said blood into a sterile container which is other than a syringe used to obtain the blood from the patient;
incubating said blood to induce production of prophylactically or therapeutically active proteins at a temperature between about 35° C. to about 39° C., wherein the incubation is conducted in the sterile container;
separating said blood into component parts by centrifugation of the blood in the sterile container;
collecting, from said component parts, a serum containing said prophylactically or therapeutically active proteins;
dividing said serum;
placing said serum into a plurality of storage containers; and
subsequently freezing said serum in said plurality of storage containers within 24 hours for storage.

2. The method of claim 1, wherein said temperature is between about 36° C. to about 38° C.

3. The method of claim 2, wherein said temperature is about 37° C.

4. The method of claim 1, wherein said incubation occurs for a period from about 6 to about 36 hours.

5. The method of claim 1, wherein said incubation occurs for a period from about 10 to about 24 hours.

6. The method of claim 1, wherein said incubation occurs for a period of about 12 hours.

7. The method of claim 1, wherein said sterile container has been modified to increase an inner surface area of the container.

8. The method of claim 7, wherein said modified sterile container is modified by treating at least part of said inner surface of said container with a corrosive agent.

9. The method of claim 7, wherein said modified sterile container is modified by adding a granulated material.

10. The method of claim 7, wherein said modified sterile container is modified by coating at least part of said inner surface of said container with an anti-coagulant.

11. A method of treating a patient with blood serum containing prophylactically or therapeutically active proteins comprising:
obtaining blood from a donor;
transferring said blood into a sterile container which is other than a syringe used to obtain the blood from the patient, wherein the sterile container is a test tube;
incubating said blood to induce production of prophylactically or therapeutically active proteins, wherein the incubation is conducted in the sterile container;
separating said blood into component parts by centrifugation of the blood in the sterile container;
collecting, from said component parts, a serum containing prophylactically or therapeutically active proteins;
dividing said serum;
placing said serum into a plurality of storage containers;
subsequently freezing said serum in said plurality of storage containers within 24 hours for storage; and
administering said serum to a patient.

12. The method of claim 11, wherein said donor and said patient are the same person.

13. The method of claim 11, wherein said donor and said patient are horses.

14. The method of claim 11, wherein said incubation is conducted at a temperature between about 35° C. to about 39° C.

15. The method of claim 11, wherein said incubation is conducted at a temperature between about 36° C. to about 38° C.

16. The method of claim 11, wherein said incubation is conducted at a temperature of about 37° C.

17. The method of claim 11, wherein said incubation occurs for a period from about 6 to about 36 hours.

18. The method of claim 11, wherein said incubation occurs for a period from about 10 to about 24 hours.

19. The method of claim 11, wherein said incubation occurs for a period of about 12 hours.

20. The method of claim 11, wherein said sterile container has been modified to increase an inner surface area.

21. The method of claim 20, wherein said modified sterile container is modified by treating at least part of said inner surface of said container with a corrosive agent.

22. The method of claim 20, wherein said modified sterile container is modified by adding a granulated material.

23. The method of claim 20, wherein said modified sterile container is modified by coating at least part of said inner surface of said container with an anti-coagulant.

24. The method of claim 1, wherein said sterile container is a test tube.
